(19) United States
(12) Patent Application Publication
(10) Pub. No.: US 2008/0039850 A1
(43) Pub. Date: Feb. 14, 2008

(54) Apparatus For Guiding A Surgical Instrument

(76) Inventors: Liam Rowley, Keighley (GB); Alex Hogg, Bardsey (GB); Michael Rock, Leeds (GB); Mark Fulton, Leeds (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/589,374

(22) PCT Filed: Mar. 7, 2005
(86) PCT No.: PCT/GB05/00861

§ 371 Date: Jul. 11, 2007

(30) Foreign Application Priority Data
Mar. 10, 2004 (GB) 0405386.4

(51) Int. Cl. A61B 17/58

(52) U.S. Cl. 606/87

(57) ABSTRACT

Apparatus for guiding a cutting tool in a surgical procedure on a bone. The apparatus includes a fixation block which can be fitted on to a bone, a guide block which can be fitted on to the fixation block, and a worm drive assembly for adjusting the orientation of the guide block rotationally relative to the fixation block about the said pivot axis. The guide block defines a path for a cutting tool, in which (i) the guide block can be translated relative to the fixation block along a translation axis so as to vary the distance between the guide block and the fixation block, and (ii) the orientation of the guide block relative to the fixation block can be adjusted lo rotationally about a pivot axis which is approximately perpendicular to the translation axis.

This invention relates to a device for guiding an instrument when performing an invasive procedure on a bone (eg a resection of a bone) and to apparatus for guiding a cutting tool in a surgical procedure on a bone.

A bone may be prepared to receive a component of an orthopaedic joint prosthesis using one or more suitable instruments. For example, a cutting instrument may be used to resect the bone so that it is appropriately shaped to fit a prosthesis. It may be important that the location and dimensions of the resection are controlled accurately to ensure a precise fit and alignment of the prosthesis on the resected bone. In order to optimise the accuracy of a resection or other preparatory step, it is known to use a guiding block which can be fixed relative to the bone. The guiding block should be positioned accurately relative to the bone and be fixed to avoid movement during the resection or other preparatory step. The guiding block can include one or more structural features which can be engaged by the instrument and which therefore provide a point of reference for locating the instrument. For example, when the instrument is a saw, the structural feature can be a surface or a pair of surfaces which define a slot against which the saw blade is moved. When the instrument is a drill, the structural feature can comprise an opening in which a drill bit can be inserted.

Fixing of a guiding block to a patient's bone is commonly achieved using bone screws or pins or other fasteners which may be inserted into pre-drilled holes or directly into the bone. The fasteners may impinge on the lateral and medial faces of the bone. The resulting configuration is relatively bulky and may cause damage to the soft tissues surrounding the bone, in particular if the bone is in part of the body that is not readily accessible. For example, during femoral resection (a preparatory step for fitting a prosthesis to a femur in knee surgery), the bulky configuration of the guiding block is such that the patella (knee cap) needs to be inverted 90° or more (typically 180°) to allow the guiding block to be fixed. Such displacement may damage the soft tissues (eg ligaments) of the knee.

The present invention seeks to improve invasive bone procedures by providing a device for guiding an instrument (eg a cutting instrument) in a guiding path adjustable through manipulation of one or more manipulators at or near to a transverse extremity of the device. In particular, the present invention relates to a device for guiding an instrument which has a guiding slot defining a guiding path which may be displaced by manipulators at or near to its lateral or medial end.

Thus according to one aspect the present invention provides a device for guiding an instrument in a guiding path when performing an invasive procedure on an extremity of a bone comprising:
a fixing block fixable on the extremity of the bone in a fixing plane;
a guiding block mountable on the fixing block substantially in the fixing plane, wherein the guiding block defines the guiding path; and
a first manipulator for manipulating the guiding path rotationally with respect to the fixing block about an axis substantially perpendicular to the fixing plane and/or a second manipulator for manipulating the guiding path substantially linearly with respect to the fixing block along an axis substantially in the fixing plane, wherein the first manipulator and/or second manipulator are manipulable from a position or positions at or near to a transverse end of the device.

The fixing block of the device of the invention can be fixed to the bone in a first step and any inaccuracy in its location can be corrected by subsequently moving the guiding block relative to the fixing block using the manipulator(s). By virtue of their position in accordance with the device of the invention, the manipulators extend out of a wound largely in a medial or lateral direction so that the procedure is advantageously less disruptive to the tissues surrounding the bone (eg the knee) and to be fitted the device requires a relatively small incision.

Preferably the extremity of the bone is a distal end and the fixing plane is a substantially distal plane. Preferably the bone is the femur.

Preferably the guiding path is a substantially planar path (ie a guiding plane).

The guiding block of the invention may comprise a guiding surface which defines the guiding path and which (for example) can be engaged by a cutting instrument such as a blade to define the appropriate alignment for a cut. When the guiding surface is planar, it will define a cutting plane. However, it can be curved or otherwise non-planar so that the bone is cut along a curved line. It might also have two parts which define separate cut lines which may intersect.

The guiding block of the invention may comprise a pair of closely spaced guide surfaces which define between them a slot in which (for example) a blade can be inserted. Preferably the slot is longer at one face of the guiding block than at the opposite face. Particularly preferably the slot is substantially trapezoidal. Preferably the slot is tapered.

The device of the invention may comprise a guiding block mountable on the fixing block substantially in the fixing plane, wherein said guiding block defines an anterior guiding path and/or a posterior guiding path. For example, the guiding block may incorporate a first slot defining an anterior guiding path and a second slot defining a posterior guiding path.

The device of the invention preferably comprises a first guiding block mountable on the fixing block substantially in the fixing plane, wherein said first guiding block defines a posterior guiding path; and a second guiding block mountable on the fixing block substantially in the fixing plane, wherein said second guiding block defines an anterior guiding path. For example, the first guiding block may incorporate a slot (eg a single slot) defining a posterior guiding path and the second guiding block may incorporate a slot (eg a single slot) defining an anterior guiding path.

In this preferred embodiment, the first guiding block and second guiding block may have opposite handedness.

The angle of the guiding path of each guiding block may be the same or different. Additional guiding blocks may be desirable (eg third and fourth guiding blocks) where (for example) the angle of the guiding path needs to be varied (eg different angles for the posterior and anterior cuts).

In a preferred embodiment the device of the invention comprises a first manipulator for manipulating the guiding path rotationally with respect to the fixing block about the axis substantially perpendicular to the fixing plane, wherein the first manipulator is manipulable from a position at or near to a transverse end of the device.

In a preferred embodiment the device of the invention comprises a second manipulator for manipulating the guiding path substantially linearly with respect to the fixing block along an axis substantially in the fixing plane, wherein the second manipulator is manipulable from a position at or near to a transverse end of the device.

In a particularly preferred embodiment the device of the invention comprises a first manipulator for manipulating the guiding path rotationally with respect to the fixing block about an axis substantially perpendicular to the fixing plane and a second manipulator for manipulating the guiding path substantially linearly with respect to the fixing block along an axis substantially in the fixing plane, wherein the first manipulator and second manipulator are manipulable from a position or positions at or near to a transverse end of the device.

The first manipulator may manipulate the guiding path rotationally about the axis of a rotational pivot shaft (eg a cylindrical rotational pivot shaft). The rotational pivot shaft may be pivotally mounted internally in the fixing block (eg in a suitable housing at or near to a transverse end of the fixing block) and secured with a pin. The first manipulator may comprise an exterior actuator connected to a stem with a portion (eg an end portion) which drives the rotational pivot shaft rotationally. For example, the end portion may be threaded (eg a helically threaded portion) and may engage an array of teeth on the exterior surface of the rotational pivot shaft so as to translate rotational manipulation of the first manipulator into rotational motion of the rotational pivot shaft about its axis. Preferably the teeth are concave parallel teeth. Preferably the rotational shaft pivot and the end portion of the first manipulator constitute a worm gear arrangement.

The second manipulator may manipulate the guiding path substantially linearly along an axis in the fixing plane by the engagement of complementary (eg male and female) threaded portions. For example, the second manipulator may comprise an exterior actuator connected to a stem with a threaded portion which engages a complementary threaded portion elsewhere (in or on the fixing block or on an internal component thereof) eg a lead screw arrangement. The threaded portion may be an intermediate portion of the stem (ie remote from the end). The non-threaded end portion is received in a bore which advantageously assists to withstand non-linear play.

Preferably the first manipulator and/or second manipulator are confined to the fixing plane. This advantageously minimises the bulkiness of the device.

Preferably the axis perpendicular to the fixing plane and the axis in the fixing plane intersect. This advantageously minimises the bulkiness of the device.

In this embodiment, the axis perpendicular to the fixing plane and the axis in the fixing plane may intersect at an axis of a rotational pivot shaft (eg a rotational pivot shaft as hereinbefore defined). The rotational pivot shaft may be pivotally mounted internally in the fixing block (eg in a suitable housing at or near to a transverse end of the fixing block) and secured with a pin. The second manipulator may manipulate the guiding path substantially linearly along an axis in the fixing plane by the engagement of complementary (eg male and female) threaded portions. Preferably the second manipulator comprises an exterior actuator connected to a stem with a threaded portion which engages a complementary threaded bore in the rotational pivot shaft.

The fixing block can be fixed to the bone by means of at least one fastener, generally two three or four fasteners. Suitable fasteners might include, for example, pins or screws. It can therefore be preferred for the fixing block to have at least one opening, preferably a plurality of openings, extending through it in which one or more fasteners can be located for fixing the fixing block to the bone.

The guiding block can be fixed to the bone by means of at least one fastener, generally two, three or four fasteners. Suitable fasteners might include, for example, pins or screws. It can therefore be preferred for the guiding block to have at least one opening, preferably a plurality of openings, extending through it in which one or more fasteners can be located for fixing the guiding block to the bone.

The guiding block may be mounted on the fixing block in any conventional manner. Preferably one or more locating pins extend between the guiding block and the fixing block. For example, one or more locating pins on the guiding block may be received in one or more apertures in the fixing block. Preferably the guiding block is confined in the fixing plane.

Of further independently patentable significance is an apparatus in which two degrees of freedom of a fixation block relative to a guide block are achieved in an advantageous manner.

In a further aspect the invention provides an apparatus for guiding a cutting tool in a surgical procedure on a bone which comprises:
a. a fixation block which can be fitted on to a bone,
b. a guide block which can be fitted on to the fixation block, the guide block defining a path for a cutting tool,
in which (i) the guide block can be translated relative to the fixation block along a translation axis so as to vary the distance between the guide block and the fixation block, and (ii) the orientation of the guide block relative to the fixation block can be adjusted rotationally about a pivot axis which is approximately perpendicular to the translation axis, and
c. a worm drive assembly for adjusting the orientation of the guide block rotationally relative to the fixation block about the said pivot axis.

Preferably the fixation block has a recess formed in it and the worm drive assembly includes a mount element which is located in the recess, in which the guide block can be fitted on to the mount element, and in which the mount element can be rotated within the recess to adjust the orientation of the guide block relative to the fixation block about the said pivot axis.

Preferably the apparatus includes an orientation adjuster which can be manipulated to adjust the rotational orientation of the guide block relative to the fixation block about the pivot axis.

Preferably the apparatus includes an orientation adjuster which can be manipulated to adjust the rotational orientation of the guide block relative to the fixation block, in which the orientation adjuster is threaded at one end and engages a surface of the mount element which is arranged approximately parallel to the axis of rotational adjustment of the guide block threadingly at or towards one end.

Preferably the translation axis and the pivot axis intersect.

Preferably the apparatus includes a connector pin which extends between the fixation block and the guide block.

Preferably the connector pin is threaded along at least a portion of its length and the guide block can be made to move along the translation axis by rotation of the connector pin.

Preferably the connector pin is threaded at or towards the end where it engages the fixation block.

Preferably the apparatus includes a first adjuster for adjusting the orientation of the guide block relative to the fixation block and a second adjuster for translating the fixation block relative to the guide block. Particularly preferably the first and second adjusters are located at or towards one end of the fixation block.

Preferably the guide block has a slot formed in it which can receive the blade of a saw.

Viewed from a yet further aspect the present invention provides a method for performing an anterior or posterior cut on a femur comprising:
(a) inserting a device as hereinbefore defined into a wound near to a distal femoral resection on the femur,
(b) fixing the fixing block onto the distal femoral resection in a distal plane;
(c) manipulating the guiding path rotationally with respect to the fixing block about an axis substantially perpendicular to the distal plane and/or manipulating the guiding path substantially linearly with respect to the fixing block along an axis substantially in the distal plane; and
(d) inserting a cutting instrument along the guiding path to perform an anterior or posterior cut on the femur.

Steps (a) to (d) may be performed advantageously without inverting the patella beyond 90°.

Steps (a) to (d) may be preceded by (i) making an incision near to the patella to define the wound and (ii) performing the distal resection on the femur.

The present invention will now be described in a non-limitative sense with reference to the accompanying Figures in which:

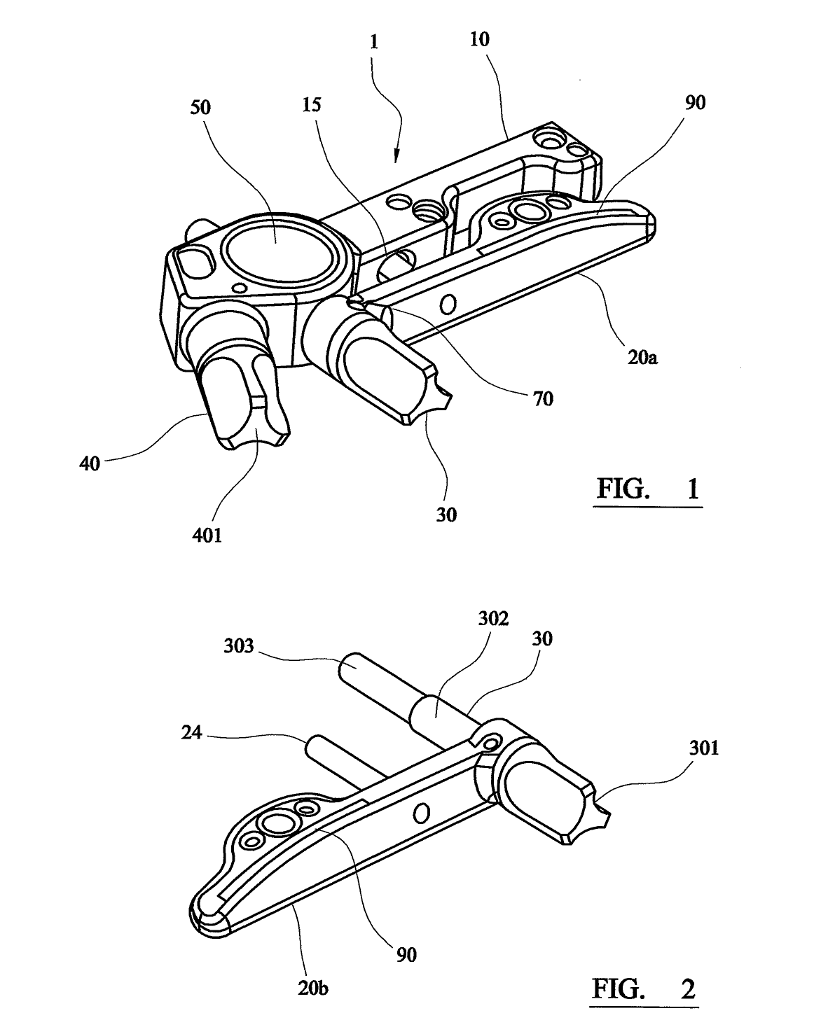

FIG. 1 illustrates a perspective view of an embodiment of the present invention with a right handed guiding block;

FIG. 2 illustrates a perspective view of an isolated left handed guiding block of an embodiment of the present invention;

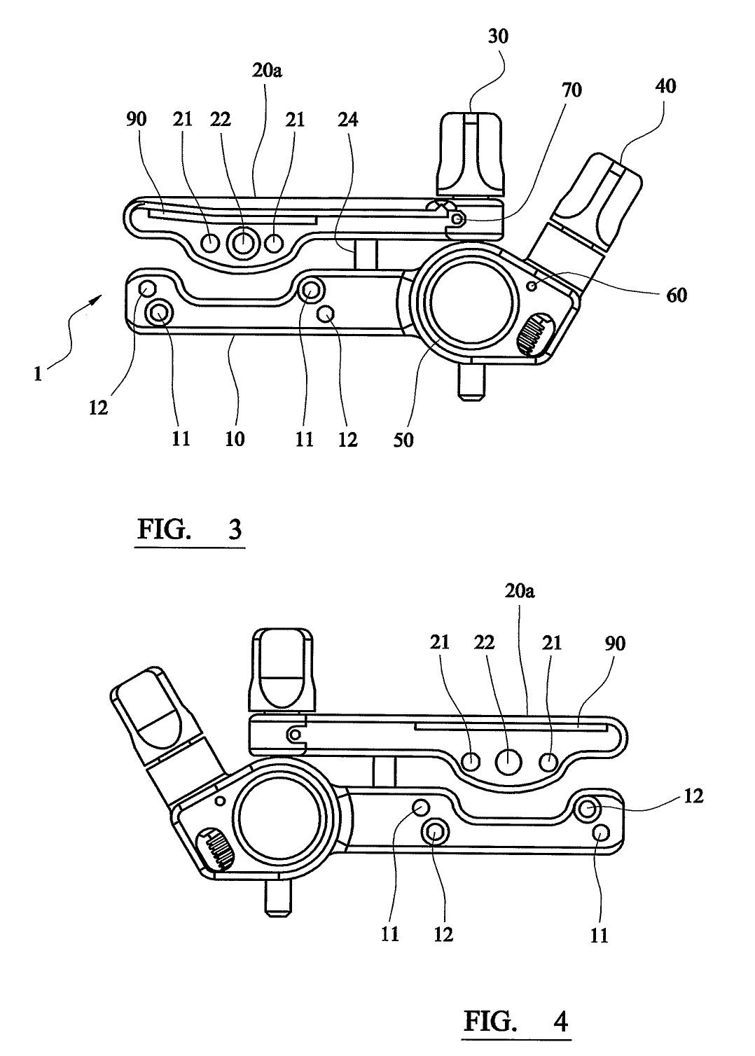

FIG. 3 illustrates an elevation of the distal (exposed) face of the embodiment of FIG. 1;

FIG. 4 illustrates an elevation of the proximal (bone-adjoining) face of the embodiment of FIG. 1; and

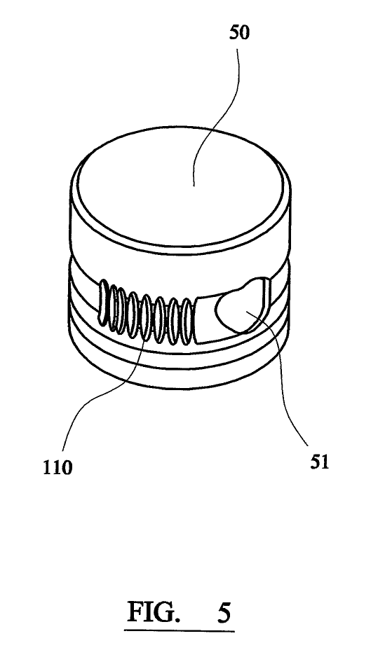

FIG. 5 illustrates a detailed perspective view of the rotational pivot shaft in isolation.

FIGS. 1-4 illustrate a first embodiment of the device of the invention designated generally by reference numeral (1). The device (1) comprises an elongate guiding block which can be right handed (20a in FIG. 1) or left handed (20b in FIG. 2 in isolation) mounted on an elongate fixing block (10). In a total knee replacement procedure, the fixing block (10) is fixed to a distally resected portion of the femur.

An aperture (not shown) at the end of the guiding block (20a,b) receives a linear manipulator (30) which itself comprises a actuator (301) at one end, a threaded portion (302) and an end portion with a substantially smooth surface (303). The guiding block (20a,b) is secured to the linear manipulator (30) by means of a pin (70) such that the actuator (301) is accessible in the distal plane at a transverse end.

A rotational pivot shaft (50) shown in isolation in FIG. 5 has a bore (51) which is threaded and complementary to the threaded portion (302) of the linear manipulator (30) on at least part of its internal surface. The bore (51) receives the threaded portion (302) and smooth portion (303) of the linear manipulator (30). The linear manipulator (30) may move linearly relative to the rotational pivot shaft (50) in the distal plane by the interaction of the male screw thread on the linear manipulator (30) with the female screw thread in the bore (51). The rotational pivot shaft (50) is mounted in a housing near to a transverse end of the fixing block (10) and secured with a pin (not seen in the Figures).

An aperture at the transverse end of the fixing block (10) receives a rotational manipulator (40) adjacent to the rotational pivot shaft (50). The rotational manipulator (40) itself comprises a actuator (401) at one end and a helically threaded portion (not seen in the Figures) at the other end. The rotational manipulator (40) is secured to the fixing block (10) by a pin (60) such that the actuator (401) is accessible in the distal plane substantially at the transverse end adjacent to the actuator (301). The helically threaded portion of the rotational manipulator (40) engages an array of concave parallel teeth (110) on the exterior surface of the rotational pivot shaft (50) so as to translate manipulation of the rotational manipulator (40) into rotational motion of the rotational pivot shaft (50) about its axis in the distal plane (ie a worm-gear arrangement).

The guiding block (20) incorporates a tapered slot (90) which is adapted to receive an instrument (eg a cutting instrument) so that a posterior or anterior resection can be made on the bone (eg femur) to which the device is fixed. The tapered slot (90) is tapered in such a way that a cutting angle is defined. This cutting angle is intended to make a cut that has a defined internal angle with the resected distal plane and thus will allow a prosthesis to be correctly fitted. The linear and rotational adjustability of the guiding block (20) allows the tapered slot (90) to be correctly positioned in the distal plane relative to the fixing block (10).

A locating pin (24) projecting from the guiding block (20a,b) is received freely in a guide aperture (15) in the fixing block (10) to confine the guiding block (20a, b) substantially in the distal plane with the fixing block (10). The guide aperture (15) is wider in the transverse axis than is the locating pin (24) allowing the locating pin (24) to move freely in the distal plane when the guiding block (20) is rotated relative to the fixing block (10) whilst withstanding any movement of the guiding block (20a,b) along the proximal/distal axis.

Whereas the fixing block (10) may be used in a left handed or right handed orientation, the guiding block (20a,b) has only one handness. The asymmetry in the guiding blocks (20a, b) is caused by the angle the tapered slot (90) makes with the fixing plane of the device. Thus if an incision is made to the right of the patella, a right-handed guiding block (20a) is required. The device is used with the actuators (301, 401) pointing out of the incision wound to the right. Consequently, if an incision is made to the left of the patella, a left-handed guiding block (20b) is required.

The fixing block (10) incorporates apertures (11, 12) to enable the fixing block (10) to be secured firmly to the surface of the bone. The apertures (11) are counterbored on one face of the fixing block (10) and the apertures (12) are counterbored on the opposite face of the fixing block (10). Thus when the fixing block is in use there will always be counterbored apertures available to receive suitable fasteners whichever orientation the fixing block (10) is in.

The guiding block (20a, b) incorporates apertures (21, 22) to enable the guiding block (20a, b) to be secured firmly to the surface of the bone once it is in the correct position. The apertures can be sized to receive a pair of pins (21) or counterbored and sized to receive a bone screw (22).

The guiding block (20a, b) narrows towards the end remote from the linear manipulator (30) to facilitate its placement posterior to the patella.

The use of the device will now be described for an incision to the right of the patella from the point of view of the surgeon (medial on the right knee, lateral on the left knee) and an incision to the left of the patella from the point of view of the surgeon (lateral on the right knee, medial on the left knee) for an anterior resection and a posterior resection. The left handed and right handed guiding blocks (20a, b) are reversed for the posterior resections ie. a left handed guiding block (20b) is used for a posterior resection with an incision to the right of the patella and a right handed guiding block (20a) is used for a posterior resection with an incision to the left of the patella.

(1) Incision to the Right of the Patella from the Point of View of the Surgeon-Anterior Resection

A right handed guiding block (20a) has been mounted on a linear manipulator (30) via a pin (70). The linear manipulator (30) is attached to the fixing block (10) by mating the threaded portion (302) of the linear manipulator (30) with the threaded aperture (51) of the rotational pivot shaft (50). The locating pin (24) of the guiding block (20a) is located in the guide aperture (15) of the fixing block (10).

An incision is made to the right of the patella and a part of the femur is resected in the distal plane using an appropriate method. During this step and subsequent steps, the patella may be displaced as little as possible to avoid damage to the delicate surrounding tissues eg the associated ligaments.

The device (1) is inserted into the wound and posterior to the patella via the incision with the actuators (301, 401) on the right extending away from the wound. The fixing block (10) is fixed to the distally resected part of the femur via suitable fasteners attached through the appropriate apertures (11). Suitable fasteners might include, for example, pins or screws. The accuracy of location of the fixing block (10) when attaching it to the resected femur need only be approximate.

Using an appropriate guiding system, the position of the tapered slot (90) required to make the correct anterior cut on the femur is assessed. The actuators (301, 401) are then used to move the guiding block (20a) relative to the fixing block (10) to position the tapered slot (90). When the actuator (301) of the linear manipulator (30) is rotated the guiding block (20a) is linearly displaced relative to the rotational pivot shaft (50). When the actuator (401) of the rotational manipulator (40) is rotated the rotational pivot shaft (50) and hence the linear manipulator (30) and guiding block (20a) is rotated relative to the fixing block (10). Therefore the guiding block (20a) and hence the tapered slot (90) can be moved to the correct position to allow the anterior cut to be made in the desired anterior cutting plane. Once in this position, the guiding block (20a) is fixed to the resected femur via suitable fasteners attached through the appropriate apertures (21, 22). Suitable fasteners might include, for example, pins or screws.

To execute the anterior cut, a cutting device such as a saw is passed through the front of the tapered slot (90) and used to resect the bone in the anterior cutting plane.

(2) Incision to the Left of the Patella from the Point of View of the Surgeon-Anterior Resection

A left handed guiding block (20b) has been mounted on a linear manipulator (30) via a pin (70). The linear manipulator (30) is attached to the fixing block (10) by mating the threaded portion (302) of the linear manipulator (30) with the threaded aperture (51) of the rotational pivot shaft (50). The locating pin (24) of the guiding block (20b) is located in the guide aperture (15) of the fixing block (10).

An incision is made to the left of the patella and a part of the femur is resected in the distal plane using an appropriate method. During this step and subsequent steps, the patella may be displaced as little as possible to avoid damage to the delicate surrounding tissues eg the associated ligaments.

The device (1) is inserted into the wound and posterior to the patella via the incision with the actuators (301, 401) on the left extending away from the wound. The fixing block (10) is fixed to the distally resected part of the femur via suitable fasteners attached through the appropriate apertures (12). Suitable fasteners might include, for example, pins or screws. The accuracy of location of the fixing block (10) when attaching it to the resected femur need only be approximate.

Using an appropriate guiding system, the position of the tapered slot (90) required to make the correct anterior cut on the femur is assessed. The actuators (301, 401) are then used to move the guiding block (20b) relative to the fixing block (10) to position the tapered slot (90). When the actuator (301) of the linear manipulator (30) is rotated the guiding block (20b) is linearly displaced relative to the rotational pivot shaft (50). When the actuator (401) of the rotational manipulator (40) is rotated the rotational pivot shaft (50) and hence the linear manipulator (30) and guiding block (20b) is rotated relative to the fixing block (10). Therefore the guiding block (20b) and hence the tapered slot (90) can be moved to the correct position to allow the anterior cut to be made in the desired anterior cutting plane. Once in this position, the guiding block (20b) is fixed to the resected femur via suitable fasteners attached through the appropriate apertures (21, 22). Suitable fasteners might include, for example, pins or screws.

To execute the anterior cut, a cutting device such as a saw is passed through the front of the tapered slot (90) and used to resect the bone in the anterior cutting plane.

(3) Incision to the Left of the Patella from the Point of View of the Surgeon-Posterior Resection

A right handed guiding block (20a) has been mounted on a linear manipulator (30) via a pin (70). The linear manipulator (30) is attached to the fixing block (10) by mating the threaded portion (302) of the linear manipulator (30) with the threaded aperture (51) of the rotational pivot shaft (50). The locating pin (24) of the guiding block (20a) is located in the guide aperture (15) of the fixing block (10).

An incision is made to the left of the patella and a part of the femur is resected in the distal plane using an appropriate method. During this step and subsequent steps, the patella may be displaced as little as possible to avoid damage to the delicate surrounding tissues eg the associated ligaments.

The device (1) is inserted into the wound and posterior to the patella via the incision with the actuators (301, 401) on the left extending away from the wound. The fixing block (10) is fixed to the distally resected part of the femur via suitable fasteners attached through the appropriate apertures (11). Suitable fasteners might include, for example, pins or screws. The accuracy of location of the fixing block (10) when attaching it to the resected femur need only be approximate.

Using an appropriate guiding system, the position of the tapered slot (90) required to make the correct posterior cut on the femur is assessed. The actuators (301, 401) are then used to move the guiding block (20a) relative to the fixing block (10) to position the tapered slot (90). When the actuator (301) of the linear manipulator (30) is rotated the guiding block (20a) is linearly displaced relative to the rotational pivot shaft (50). When the actuator (401) of the rotational manipulator (40) is rotated the rotational pivot shaft (50) and hence the linear manipulator (30) and guiding block (20a) is rotated relative to the fixing block (10). Therefore the guiding block (20a) and hence the tapered slot (90) can be moved to the correct position to allow the posterior cut to be made in the desired posterior cutting plane. Once in this position, the guiding block (20a) is fixed to the resected femur via suitable fasteners attached through the appropriate apertures (21, 22). Suitable fasteners might include, for example, pins or screws.

To execute the posterior cut, a cutting device such as a saw is passed through the front of the tapered slot (90) and used to resect the bone in the posterior cutting plane.

(4) Incision to the Right of the Patella from the Point of View of the Surgeon-Posterior Resection

A left handed guiding block (20b) has been mounted on a linear manipulator (30) via a pin (70). The linear manipulator (30) is attached to the fixing block (10) by mating the threaded portion (302) of the linear manipulator (30) with the threaded aperture (51) of the rotational pivot shaft (50). The locating pin (24) of the guiding block (20b) is located in the guide aperture (15) of the fixing block (10).

An incision is made to the right of the patella and a part of the femur is resected in the distal plane using an appropriate method. During this step and subsequent steps, the patella may be displaced as little as possible to avoid damage to the delicate surrounding tissues eg the associated ligaments.

The device (1) is inserted into the wound and posterior to the patella via the incision with the actuators (301, 401) on the right extending away from the wound. The fixing block (10) is fixed to the distally resected part of the femur via suitable fasteners attached through the appropriate apertures (12). Suitable fasteners might include, for example, pins or screws. The accuracy of location of the fixing block (10) when attaching it to the resected femur need only be approximate.

Using an appropriate guiding system, the position of the tapered slot (90) required to make the correct posterior cut on the femur is assessed. The actuators (301, 401) are then used to move the guiding block (20b) relative to the fixing block (10) to position the tapered slot (90). When the actuator (301) of the linear manipulator (30) is rotated the guiding block (20b) is linearly displaced relative to the rotational pivot shaft (50). When the actuator (401) of the rotational manipulator (40) is rotated the rotational pivot shaft (50) and hence the linear manipulator (30) and guiding block (20b) is rotated relative to the fixing block (10). Therefore the guiding block (20b) and hence the tapered slot (90) can be moved to the correct position to allow the posterior cut to be made in the desired posterior cutting plane. Once in this position, the guiding block (20b) is fixed to the resected femur via suitable fasteners attached through the appropriate apertures (21, 22). Suitable fasteners might include, for example, pins or screws.

To execute the posterior cut, a cutting device such as a saw is passed through the front of the tapered slot (90) and used to resect the bone in the posterior cutting plane.

1. Apparatus for guiding a cutting tool in a surgical procedure on a bone which comprises:
a. a fixation block which can be fitted on to a bone,
b. a guide block which can be fitted on to the fixation block, the guide block defining a path for a cutting tool, wherein (i) the guide block can be translated relative to the fixation block along a translation axis so as to vary the distance between the guide block and the fixation block, and (ii) the orientation of the guide block relative to the fixation block can be adjusted lo rotationally about a pivot axis which is approximately perpendicular to the translation axis, and
c. a worm drive assembly for adjusting the orientation of the guide block rotationally relative to the fixation block about the said pivot axis.

2. The apparatus of claim 1, wherein the fixation block has a recess formed in it, the worm drive assembly includes a mount element located in the recess, the guide block can be fitted on to the mount element, and the mount element can be rotated within the recess to adjust the orientation of the guide block relative to the fixation block about the said pivot axis.

3. The apparatus of claim 1, further comprising which an orientation adjuster that can be manipulated to adjust the rotational orientation of the guide block relative to the fixation block about the pivot axis.

4. The apparatus of claim 2, further comprising an orientation adjuster that can be manipulated to adjust the rotational orientation of the guide block relative to the fixation block, wherein the orientation adjuster is threaded at one end and engages a surface of the mount element which is arranged approximately parallel to the axis of rotational adjustment of the guide block threadingly at or towards one end.

5. The apparatus of claim 1, wherein the translation axis and the pivot axis intersect.

6. The apparatus of claim 1, further comprising a connector pin which extends between the fixation block and the guide block.

7. The apparatus of claim 6, wherein the connector pin is threaded along at least a portion of its length, and wherein the guide block can be made to move along the translation axis by rotation of the connector pin.

8. The apparatus of claim 7, wherein the connector pin is threaded remote from the end where the connector pin engages the fixation block.

9. The apparatus of claim 1, further comprising a first adjuster for adjusting the orientation of the guide block relative to the fixation block and a second adjuster for translating the fixation block relative to the guide block.

10. The apparatus of claim 9, wherein the first and second adjusters are located at or towards one end of the fixation block.

11. The apparatus of claim 1, wherein the guide block has a slot formed therein configured to receive the blade of a saw.

12. A device for guiding an instrument in a guiding path when performing an invasive procedure on an extremity of a bone comprising:
a fixing block fixable on the extremity of the bone in a fixing plane;
a guiding block mountable on the fixing block substantially in the fixing plane, wherein the guiding block defines the guiding path; and
a first manipulator for manipulating the guiding path rotationally with respect to the fixing block about an axis substantially perpendicular to the fixing plane and/or a second manipulator for manipulating the guiding path substantially linearly with respect to the fixing block along an axis substantially in the fixing plane, wherein the first manipulator and/or second manipulator are manipulable from a position or positions at or near to a transverse end of the device.

13. The device of claim 12, wherein the guiding path is a substantially planar path.

14. The device of claim 12, further comprising a first guiding block mountable on the fixing block substantially in the fixing plane, the first guiding block defining a posterior guiding path, and a second guiding block mountable on the fixing block substantially in the fixing plane, the second guiding block defining an anterior guiding path.

15. The device of claim 14, wherein the first guiding block and second guiding block have opposite handedness.

16. The device of claim 12, further comprising a first manipulator for manipulating the guiding path rotationally with respect to the fixing block about the axis substantially perpendicular to the fixing plane, the first manipulator being manipulable from a position at or near to a transverse end of the device.

17. The device of claim 12, further comprising a second manipulator for manipulating the guiding path substantially linearly with respect to the fixing block along an axis substantially in the fixing plane, the second manipulator being manipulable from a position at or near to a transverse end of the device.

18. The device of claim 12, further comprising a first manipulator for manipulating the guiding path rotationally with respect to the fixing block about an axis substantially perpendicular to the fixing plane and a second manipulator for manipulating the guiding path substantially linearly with respect to the fixing block along an axis substantially in the fixing plane, the first manipulator and second manipulator being manipulable from a position or positions at or near to a transverse end of the device.

19. The device of claim 12, wherein the first manipulator manipulates the guiding path rotationally about the axis of a rotational pivot shaft.

20. The device of claim 19, wherein the rotational pivot shaft is pivotally mounted internally in the fixing block.

21. The device of claim 18, wherein the first manipulator comprises an exterior actuator connected to a stem with an end portion which drives the rotational pivot shaft rotationally.

22. The device of claim 21, wherein the first manipulator translates rotational manipulation of the exterior actuator into rotational motion of the rotational pivot shaft about its axis.

23. The device of claim 21, wherein the end portion is threaded and engages an array of teeth on the exterior surface of the rotational pivot shaft so as to translate rotational manipulation of the exterior actuator into rotational motion of the rotational pivot shaft about its axis.

24. The device of claim 23, wherein the teeth are concave parallel teeth.

25. The device of claim 12, wherein the second manipulator manipulates the guiding path substantially linearly along an axis in the fixing plane by the engagement of complementary threaded portions.

26. The device of claim 25, wherein the second manipulator comprises an exterior actuator connected to a stem with a threaded portion which engages a complementary threaded portion.

27. The device of claim 12, wherein the axis substantially perpendicular to the fixing plane and the axis in the fixing plane intersect.

28. The device of claim 27, wherein the axis substantially perpendicular to the fixing plane and the axis in the fixing plane intersect at an axis of a rotational pivot shaft.

29. The device of claim 28, wherein the second manipulator comprises an exterior actuator connected to a stem with a threaded portion, the threaded portion engaging a complementary threaded bore in the rotational pivot shaft.
